(19) United States
(12) Patent Application Publication
(10) Pub. No.: US 2016/0324178 A1
(43) Pub. Date: Nov. 10, 2016

(54) Structuring and Gelling Agents

(71) Applicant: ARCHER DANIELS MIDLAND COMPANY, Decatur, IL (US)

(72) Inventors: Kenneth Stensrud, Decatur, IL (US); Shireen Baseeth, Decatur, IL (US); Swapnil Jadhav, Bloomington, IL (US); John Inmok Lee, Decatur, IL (US); Lori Wicklund, Argenta, IL (US)

(21) Appl. No.: 15/105,771

(22) PCT Filed: Dec. 18, 2014
(86) PCT No.: PCT/US14/71066

§ 371 Date: Jun. 17, 2016

Related U.S. Application Data
(60) Provisional application No. 61/919,331, filed on Dec. 20, 2013.

(51) Int. Cl. A23D 9/007; A23L 29/20; C12P 19/02

(52) U.S. Cl. CPC A23D 9/007; C12P 19/02; C12Y 301/01003; A23L 29/20; A23V 2002/00

(57) ABSTRACT

Structured, non-polar liquid oleogels comprising non-polar liquids and a derivative of an anhydrohexitol are disclosed. Processes for producing such structured, non-polar liquid oleogels are also disclosed. Also disclosed are processes for producing esters of 1,4:3,6-dianhydrohexitol.

### CROSS-REFERENCE TO RELATED APPLICATIONS

This application claims priority to U.S. Provisional Patent Application No. 61/919,331, filed Dec. 20, 2013, the contents of the entirety of which is incorporated by this reference.

### TECHNICAL FIELD

The present invention relates generally to structuring and gelling agents, and more particularly to the production and use of a derivative of an anhydrohexitol as such structuring or gelling agents.

### BACKGROUND OF THE INVENTION

Non-polar liquids (e.g., organics, solvents, ionic fluids, and oils) are present in a large number of products used by humans every day. Such non-polar liquids are fluid-like and are often incorporated into products as structured liquids or liquids that have enhanced mechanical properties. Structuring of such liquids is a process that involves physically transforming a liquid into a more solid material. Such structuring may also enhance the texture, appearance, taste, and stability of the non-polar liquids, depending on the ultimate use of such liquids. Of the various non-polar liquids, oils are often subjected to a structuring process for use in developing functional products such as foods, cosmetics, pharmaceuticals, lubricants, and use in other industries.

Oils are often structured by the addition of high-melting point saturated or trans fats such as saturated triglycerides, hardstocks, long chain saturated fatty acids, or others. Such structuring components may crystallize and form a fat crystal network when incorporated in the oil. The liquid oil becomes entrapped in such crystalline network and converts the oil from the liquid phase to a semi-solid plastic material.

Another way to structure oil is by organogelation. The organogelation process involves the addition of self-assembling molecules or organogelators into an oil phase. The organogelators self-assemble through intermolecular non-covalent interactions (e.g., hydrogen bonding, van der Waals forces, or London dispersion forces) to form a three dimensional fibrous network that mimics a fat crystal network. Similar to the fat crystal network, the fibrous network entraps and immobilizes oil entrapped within such fibrous network. Oils structured with organogelators may be referred to as organogels and typically exhibit viscoelastic and/or thermoreversible properties.

Organogelators are surfactant-like low molecular weight molecules (i.e., less than 3000 daltons) and are able to structure oils at very low concentrations (about 2-4% by weight). The molecular structure of organogelators may be manipulated to produce organogels with tailor-made properties. The organogelators have been explored for use in food applications as alternatives to saturated fats in order to improve the nutritional profile of the foods. Organogelators may also find utility in foods (e.g., structuring liquid oils), pharmaceuticals (e.g., control release vehicles for bioactives), cosmetics (e.g., structuring of lipsticks, moisturizers, or sunscreens), in remediation (e.g., structuring of used cooking oil), and lubricants. Some organogelators that have been used are fatty alcohols, amino acid amides, sorbitan alkylates, and phytochemicals.

While structuring and gelling agents exist, needs exist for new, biobased structuring agents having different functionalities.

### SUMMARY OF THE INVENTION

In each of its various embodiments, the present invention fulfills this need and discloses derivatives of anhydrohexitols, processes for producing such derivatives of anhydrohexitols, and uses of such derivatives of anhydrohexitols.

In one embodiment, a structured non-polar liquid oleogel comprises a non-polar liquid and a derivative of an anhydrohexitol.

In another embodiment, a method of producing a structured non-polar liquid comprises mixing a derivative of an anhydrohexitol with the non-polar liquid, thus producing a mixture, and heating the mixture.

In yet a further embodiment, a process for producing an ester of a 1,4:3,6-dianhydrohexitol comprises combining the 1,4:3,6-dianhydrohexitol with a carboxylic acid, thus producing a mixture. The process further includes placing the mixture in contact with an enzyme, such that an ester bond is formed between the 1,4:3,6-dianhydrohexitol and the carboxylic acid.

### DESCRIPTION OF THE DRAWINGS

FIG. 1 illustrates one embodiment of a network of agglomerated derivatives of anhydrohexitols of the present invention.

FIG. 2 shows the ability of various derivatives of anhydrohexitols of the present invention to form a gel in a liquid oil.

FIG. 3 shows the stress point at which gels formed by derivatives of anhydrohexitols of the present invention in liquid oils begin flowing as a fluid.

FIG. 4 shows the ability of various derivatives of anhydrohexitols of the present invention to form a gel in a liquid oil.

FIG. 5 shows the stress point at which gels formed by derivatives of anhydrohexitols of the present invention in liquid oils begin flowing as a fluid.

FIG. 6 is one embodiment of a structure of a derivative of an anhydrohexitol of the present invention.

FIG. 7 is another embodiment of a structure of a derivative of an anhydrohexitol of the present invention.

FIG. 8 shows the ability of various derivatives of anhydrohexitols of the present invention to form a gel as compared to sorbitan monoesters.

FIG. 9 shows the stress point at which gels formed by various derivatives of anhydrohexitols of the present invention begin flowing as a fluid as compared to sorbitan monoesters.

FIG. 10 shows various parameters used to determine the critical packing parameter of various chemical structures.

### DETAILED DESCRIPTION OF THE INVENTION

In one embodiment, a non-polar liquid structured with a derivative of an anhydrohexitol is disclosed. The derivative may be an ester of the anhydrohexitol, an amide of the anhydrohexitol, an ether of the anhydrohexitol, a dimer of the anhydrohexitol, or combinations of any thereof.

In a further embodiment, the non-polar liquid of the structured non-polar liquid oleogel of the present invention may have a dielectric constant of between 0 and 40 or in another embodiment, a dielectric constant of less than 20.

The derivatives of anhydrohexitols of the present invention may be used to form fibrilar network gels in long chain hydrocarbon solvents. Such gels may find utility in applications for use in foods, colorants for paints and/or inks, as lubricants, as oil binding additives in cosmetics (i.e., lipstick), in dermatological applications, or personal care products (i.e., antiperspirants). The gels may also find utility in plant protection formulations or seed coating applications. The gels may also be used in detergents or cleaners.

In a further embodiment, gels formed with the derivatives of anhydrohexitols are thermoreversible in that they are liquid at higher temperatures and gels at lower temperatures. For instance, the structured non-polar liquid oleogel may melt at a temperature of at least 40° C. and re-form to the shape of a gel at a temperature of less than 40° C. The structured non-polar liquid oleogels may contain between about 0.1-15% by weight of the derivative of the anhydrohexitol or between about 2-10% of the derivative of the anhydrohexitol by weight. The non-polar liquid may be present in the structured non-polar liquid oleogel at an amount of between about 80-99.9% by weight.

In a further embodiment, the derivative of the anhydrohexitol may be used as a structuring agent for an organic liquid for use in cosmetics, pharmaceuticals, food, lubricant, or other industrial product. The derivative of the anhydrohexitol may form a self-assembled, liquid crystalline structure in the non-polar liquid.

In yet an additional embodiment, the structured non-polar liquid oleogel may further comprising a structuring agent. Such structuring agent may include, without limitation, ethyl cellulose, hydroxyl-stearic acid, sterols, proteins, emulsifiers, waxes, or combinations of any thereof.

In another embodiment, the derivative of the anhydrohexitol may be used to structure non-polar compounds and function as a lubricant, grease, coolant, or other industrial compound, as no modification of the organic compounds is required. The structured organic compounds will not exhibit any coarse consistency making such structured organic compounds useful for tribological applications.

In a further embodiment, the derivative of the anhydrohexitol may be used as primary or secondary emulsifiers. In another embodiment, the structured non-polar liquid oleogels may further comprise an emulsifier. Non-limiting examples of emulsifiers that may be used include anionic surfactants including, but not limited to, sodium and potassium salts of straight-chain fatty acids, polyoxyethylenated fatty alcohol carboxylates, linear alkyl benzene sulfonates, alpha olefin sulfonates, sulfonated fatty acid methyl ester, arylalkanesulfonates, sulfosuccinate esters, alkyldiphenylether(di)sulfonates, alkylnaphthalenesulfonates, isoethionates, alkylether sulfates, sulfonated oils, fatty acid monoethanolamide sulfates, polyoxyethylene fatty acid monoethanolamide sulfates, aliphatic phosphate esters, nonylphenolphosphate esters, sarcosinates, fluorinated anionics, anionic surfactants derived from oleochemicals, and combinations of any thereof. Non-limiting examples of non-ionic surfactants that may be used as the emulsifier include, but are not limited to, sorbitan monostearate, polyoxyethylene ester of rosin, polyoxyethylene dodecyl mono ether, polyoxyethylene-polyoxypropylene block copolymer, polyoxyethylene monolaurate, polyoxyethylene monohexadecyl ether, polyoxyethylene monooleate, polyoxyethylene mono(cis-9-octadecenyl)ether, polyoxyethylene monostearate, polyoxyethylene monooctadecyl ether, polyoxyethylene dioleate, polyoxyethylene distearate, polyoxyethylene sorbitan monolaurate polyoxyethylene sorbitan monooleate, polyoxyethylene sorbitan monopalmitate, polyoxyethylene sorbitan monostearate, polyoxyethylene sorbitan trioleate, polyoxyethylene sorbitan tristearate, polyglycerol ester of oleic acid, polyoxyethylene sorbitol hexastearate, polyoxyethylene monotetradecyl ether, polyoxyethylene sorbitol hexaoleate, fatty acids, tall-oil, sorbitol hexaesters, ethoxylated castor oil, ethoxylated soybean oil, rapeseed oil ethoxylate, ethoxylated fatty acids, ethoxylated fatty alcohols, ethoxylated polyoxyethylene sorbitol tetraoleate, glycerol and polyethylene glycol mixed esters, alcohols, polyglycerol esters, monoglycerides, sucrose esters, alkyl polyglycosides, polysorbates, fatty alkanolamides, polyglycol ethers, derivatives of any thereof, and combinations of any thereof.

In other embodiments, the compositions of the present invention may be used to solubilize polar, non-polar, and/or amphiphilic guest molecules. In another embodiment, the compositions of the present invention may be used to solubilize or carry enzymes.

In another embodiment, the compositions of the present invention may be used in a food product. In such embodiments, non-limiting uses of the composition include, without limitation: a structuring agent for providing or enhancing structure in foods such as, for example, in spreads, mayonnaise, dressing, sauce, shortenings, fluid oils, high-oleic oils, fillings, icings, frostings, a creamer, compound coatings, chocolate, confectionary chips, confectionary chunks, an emulsifier that can be used to carry active ingredients or enzymes such as in baking applications, a film forming composition that can hold active ingredients, a coating for carrying spices, seasonings or flavorings on a food, a film-forming composition that could be used as a release agent, a beverage emulsion, or as a carrier for delivering nutritional or bio-active compounds.

In one embodiment, the compositions of the present invention may also be used to partially substitute for or replace saturated fats in edible oils to improve the nutritional profile, yet still provide the same functionality as the saturated fats.

In a further embodiment, the compositions of the present invention may be used to develop jelly emulsions (or emulsion gels), such as in a organogel-hydrogel heterogeneous mixture.

In one embodiment, the compositions of the present invention may be used in the cocoa industry to produce chocolate, cocoa containing foods, chocolate drops, compound drops, wafers, compound coatings, chocolate coatings, coating products, chips, chunks, white chocolate, other confectionary products, or function as a cocoa butter equivalent. The composition may also be used to partially substitute for or replace cocoa butter in cocoa products to improve the nutritional profile. In another embodiment, the compositions may be used in conjunction with cocoa powder, cocoa butter, cocoa liquor, a vanilla flavoring other flavorings, a sweetener, a vegetable fat, or combinations of any thereof in the production a confectionary product.

In one embodiment, the compositions of the present invention may be used to: improve the stability of an active ingredient; function as an emulsion stabilizer; lower the saturated fat content of a food and/or produce a food having a low saturated fat content; carry polar antioxidants; improve the pliability of a fat in puff pastry; improve the spreadability of a high protein product such as a crème filling; decrease the usage level of an emulsion; replace trans fat in a food product; produce a lower fat product; or other uses.

In one embodiment, the non-polar liquid of the present invention comprises vegetable oil such as triglyceride and/or diglyceride oils, a food-grade low hydrophilic lipophilic balance (HLB) emulsifier, polyol esters, monoglycerides, diglycerides, fatty acid esters, or combinations of any thereof. The non-polar liquid may be soybean oil, canola oil, sunflower oil, olive oil, sesame oil, grapeseed oil, rapeseed oil, linseed oil, neem oil, liquid paraffin, corn oil, soy polyols, biodiesel, diesel oil, a lubricant grease, a modified vegetable oil, a petroleum based solvent, mineral oil, esters of any thereof, and combination of any thereof.

In an additional embodiment, each of the components of the compositions of the present invention is edible and/or approved for use in foods. In a further embodiment, a preservative may added to the compositions of the present invention for use in foods. Examples of preservatives include, but are not limited to, potassium sorbate, citric acid, sodium benzoate, or other good grade preservatives.

In a further embodiment, the compositions of the present invention may be configured as a food ingredient for use in a food stuff, beverage, nutraceutical, pharmaceutical, pet food, or animal feed. In one embodiment, the composition of the present invention may further comprise a compound selected from the group consisting of green tea extract, a flavoring agent, ascorbic acid, potassium sorbate, citric acid, natural polar antioxidants, tocopherols, sterols or phytosterols, saw palmetto, caffeine, sea weed extract, grape-seed extract, rosemary extract, almond oil, lavender oil, peppermint oil, bromelain, capsaicin, emulsifiers, or combinations of any thereof.

In one embodiment, the compositions described herein are bio-based. Bio-based content of a product may be verified by ASTM International Radioisotope Standard Method D 6866. ASTM International Radioisotope Standard Method D 6866 determines bio-based content of a material based on the amount of bio-based carbon in the material or product as a percent of the weight (mass) of the total organic carbon in the material or product. Bio-derived and bio-based products will have a carbon isotope ratio characteristic of a biologically derived composition.

It should be understood that this invention is not limited to the embodiments disclosed in this summary, or the description that follows, but is intended to cover modifications that are within the spirit and scope of the invention, as defined by the claims.

Other than in the examples described herein, or unless otherwise expressly specified, all of the numerical ranges, amounts, values and percentages, such as those for amounts of materials, elemental contents, times and temperatures of reaction, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains error necessarily resulting from the standard deviation found in its underlying respective testing measurements. Furthermore, when numerical ranges are set forth herein, these ranges are inclusive of the recited range end points (i.e., end points may be used). When percentages by weight are used herein, the numerical values reported are relative to the total weight.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10. The terms “one,” “a,” or “an” as used herein are intended to include “at least one” or “one or more,” unless otherwise indicated

In an embodiment, an derivative of an anhydrohexitol may be 1,4:3,6-dianhydrohexitol. In other embodiments, the anhydrohexitol may be isosorbide, isomannide, or isoiodide which are natural building blocks that are highly stable and possess two functional hydroxyl groups that are amenable to chemical modification. The 1,4:3,6-dianhydrohexitols have emerged as a versatile platform to synthesize chemicals having potential applications as high boiling point solvents, pre-cursors for polymers, fuel additives, or surfactants.

In one embodiment, the present invention discloses an organogelator or structuring agent that can be produced from an anhydrohexitol, such as a 1,4:3,6-dianhydrohexitol. In another embodiment, a derivative of an anhydrohexitol such as 1,4:3,6-dianhydrohexitol is produced which finds utility as a gelator in a non-polar liquid. Non-polar liquids which may be structured include, but are not limited to, edible oils, non-edible oils, essential oils, fish oils, and paraffinic oils.

With the increasing commercial demand of renewable resources to replace non-renewable resources for various economic and environmental concerns, processes and products that use sustainable technology has become more important. Thus, the derivatives of the anhydrohexitols of the present invention may be produced using green processes to sustainably produce value-added products.

In another embodiment, a method of producing a structured non-polar liquid comprises mixing a derivative of an anhydrohexitol with the non-polar liquid, thus producing a mixture, and heating the mixture. The mixture may also be agitated during heating. The method may also include cooling the heated, mixture, thus forming a gel. An emulsifier may also be added to the mixture.

In one embodiment, the derivatives of the anhydrohexitols may be produced using lipase, one of nature's readily available catalysts. The derivatives of the anhydrohexitols may be produced by conjugating a carboxylic acid moiety to one or both of the hydroxyl groups on the anhydrohexitol via an ester linkage, thus producing a mono- or di-ester derivative of the anhydrohexitol. The resultant mono- or di-ester derivative of the anhydrohexitol may be purified using various chromatographic techniques, distillation, or other known processes.

The production of the derivatives of the anhydrohexitols of the present invention may include combing the anhydrohexitol with a carboxylic acid, thus producing a mixture and placing the mixture in contact with an enzyme (e.g., lipase), such that an ester bond is formed between the anhydrohexitol and the carboxylic acid. The mixture may further include an organic solvent. In one embodiment, water may be removed from the mixture such that ester bond formation occurs in the absence of water.

In one embodiment, the carboxylic acid moiety that may be attached to the anhydrohexitol may be a fatty acid. The fatty acids may range from having one carbon, i.e., formic acid, up to 20 carbons, i.e., arachidic acid and in another embodiment, the fatty acid may have from 18 to 20 carbons. In other embodiments, the carboxylic acid may selected from the group consisting of unsaturated monocarboxylic acids, amino acids, keto acids, aromatic carboxylic acids, dicarboxylic acids, tricarboxylic acids, alpha hydroxyl acids, saturated fatty acids, or combinations of any thereof. The carboxylic acid may also be selected from the group consisting of formic acid, acetic acid, chloroacetic acid, dichloroacetic acid, trichloroacetic acid, oxalic acid, benzoic acid, or combinations of any thereof. In an additional embodiment, the carboxylic acid moiety is a biobased compound.

In another embodiment, the anhydrohexitol may be selected from the group consisting of isosorbide, isomannide, isoiodide, or combinations of any thereof. In a further embodiment, the anhydrohexitol is a 1,4:3,6-dianhydrohexitol.

Uses of a derivative of an anhydrohexitol as a plasticizer are also disclosed. The derivative of the anhydrohexitol may be selected from the group consisting of an ester of the anhydrohexitol, an amide of the anhydrohexitol, an ether of the anhydrohexitol, a dimer of the anhydrohexitol, or combinations of any thereof. The ester of the anhydrohexitol may be a diester. The derivative of the anhydrohexitol may be a 1,4:3,6-dianhydrohexitol selected from the group consisting of isosorbide, isomannide, isoiodide, or combinations of any thereof.

The present invention may be further understood by reference to the following examples. The following examples are merely illustrative of the invention and are not intended to be limiting. Unless otherwise indicated, all parts are by weight. Examples.

### EXAMPLE 1

Enzymatic synthesis of 1,4:3,6:dianhydrohexitols-fatty acid esters in the presence of solvent.

8.2 grams (56 mmol) of purified isosorbide (i.e., greater than 99% pure) was mixed with 41 g of acetone, thus forming a one-phase solution. 28.2 g (100 mmol) technical-grade oleic acid obtained from Sigma-Aldrich, St. Louis, Mo., was added to the isosorbide/acetone mixture and the resultant mixture remained as a one-phase solution. 6 g of LIPOZYME 434 brand lipase, available from Novozymes, Franklinton, N.C., was mixed into the one-phase solution at the start of a reaction in a shaker flask rotating at 180 rpm and maintained 40° C.

Samples of the reaction mixture were taken after 18 hours and after 42 hours. 1 microliter of the samples were analyzed on a thin layer chromatography (TLC) plate, a Whatman Partisil K6, 5×10 cm, silica gel 60 Å plate with a thickness of 250 μm. A mixture of hexane/ethyl ether at a ratio of 1/1 was used to develop the TLC plate. The developed plate was sprayed with 5% sulfuric acid in methanol and placed in a 110° C. oven for darkening spots on the plate. A majority of the oleic acid remained non-reacted which indicated an inefficient esterification reaction, even after 42 hours. It was though that an accumulation of water in the reaction mixture was the cause of the ineffective esterification reaction.

### EXAMPLE 2

Enzymatic synthesis of 1,4:3,6:dianhydrohexitols-fatty acid esters in the presence of solvent and absence of water.

The reaction of Example 1 was repeated, except a 25 g molecular sieve (e.g., 3Å, available from Hongye Chemical, Shanghai, China) was used to remove moisture. Almost all of the fatty acids were esterified after 16 hours of reaction time at 40° C. This example demonstrates the importance of removing water from the reaction.

### EXAMPLE 3

Enzymatic synthesis of 1,4:3,6:dianhydrohexitols-fatty acid esters in the absence of solvent and water.

Isosorbide has a melting point of between 60-63° C. and melted isosorbide has a fair solubility in fatty acid at elevated temperature. LIPOZYME 435 brand lipase is reasonably stable at elevated temperature and low moisture. Such conditions led to the concept of reacting straight isosorbide and fatty acids at an elevated temperature under vacuum to remove the water generated from the esterification reaction.

84.6 g (300 mmol) of oleic acid was placed in a 250 ml round bottom flask with 3 necks and heated to 80° C. under vacuum at about 5 ton. 11.4 g (10% by weight on total substrate) of LIPOZYME 435 brand lipase was mixed into the oleic acid and agitated for about 5 minutes at 80° C. 29.2 g (200 mmol) of isosorbide (99% purity) was added to the flask before pulling vacuum at about 5 torr. The reaction was allowed to continue under vacuum at 80° C. at about 350 rpm of agitation for 14 hours.

To analyze the reaction product, one part of the reaction product was mixed with 10 parts hexane and 2 μl of the reaction product/hexane mixture was spotted on a TLC plate. TLC analysis of the reaction product showed that the reaction was completed with almost of the oleic acid being gone. This example shows that the reaction can proceed without solvent.

### EXAMPLE 4

Esterification with less pure isosorbide.

The reaction conditions of Example 3 were followed with the following modifications: 32.8 g (225 mmol) of isosorbide (greater than 95% purity) and 67.7 g (240 mmol) of oleic acid were used. The progress of the reaction was monitored by measuring residual free fatty acids (FFA) in the reaction mixture using AOCS method Ca 5a-40. The amount of residual FFAs present after 1 hour was 3.3% and 0.7% after 2 hours, indicating that the reaction was complete in less than 2 hours. Mono- and di-esters of the 1,4:3,6-dianhydrohexitol were visible on the TLC plate.

### EXAMPLE 5

Esterification of oleic acid with various enzymes.

LIPOZYME RM IM brand lipase and LIPOZYME TL IM brand lipase show preference on hydroxyl groups at different positions on a glycerol backbone. Isosorbide is a bridged-ring structured molecule and has both endo- and exo-hydroxyl isomers. The two lipases were tested to see if there was a preference towards a particular hydroxyl group on the isosorbide, which could preferentially produce mono-esters.

67.7 g (240 mmol) of oleic acid was heated to 50° C. under vacuum. 10 g (10% by weight on total substrate dosage) of LIPOZYME RM IM brand lipase was added to the oleic acid and agitated for 5 minutes, at which point 32.8 g (225 mmol) of 95% purity isosorbide was added to the reaction flask. The reaction was performed under vacuum with about 350 rpm mixing and samples were taken after 2 hours and 19 hours. The samples were assayed for free fatty acid reduction. After 2 hours, there were 89% free fatty acids and after 19 hours, there were 81% fatty acids indicating a slow esterification reaction. However, only mono-ester derivatives of 1,4:3,6-dianhydrohexitol were detected in the reaction mixture and no di-ester derivatives of 1,4:3,6-dianhydrohexitol were detected. The mono-ester derivatives of 1,4:3,6-dianhydrohexitol were identified as endo-hydroxyl isomer indicating a preference of the endo-hydroxyl by the LIPOZYME RM IM brand lipase.

### EXAMPLE 6

Esterification of oleic acid with various enzymes.

In this example, LIPOZYME TL IM brand lipase was evaluated for its ability for isosorbide esterification. The reaction was run substantially the same as Example 5, except the reaction temperature was 80° C. and LIPOZYME TL IM brand lipase was used.

Samples were taken from the reaction mixture at 1 and 4 hours and analyzed for free fatty acid content. At 1 hour, the free fatty acid content was 93% and at 4 hours, the free fatty acid content was 96%. The identity of the formed esters was not performed due to the slow reaction.

### EXAMPLE 7

Production and purification of isosorbide fatty acid esters by short-path still.

282 g (1 mole) of oleic acid, 42.8 g of LIPOZYME 435 brand lipase, and 146 g (1 mole) of isosorbide were used. The reaction was carried out at 80° C. substantially as described in Example 3. The reaction achieved 0.56% free fatty acids after 3 hours.

The resultant reaction mixture was filtered over Whatman #40 filter paper to remove the enzyme. The reaction mixture was tested by TLC plate for the presence of mono- and di-esters and both were determined to be present. Short-path distillation was tested to separate the di-esters from the mono-esters and to remove any residual fatty acids. 250 g of the reaction mixture was processed at 210° C., 230 rpm rotor speed, and about 0.024 mb vacuum. The flow rate was set at about 6 ml/min. The retentate fraction was 105 g and had 0.1% FFA remaining TLC plating showed the retentate fraction was exclusively di-esters with no presence of mono-esters. The distillate fraction was 129 g and had 0.9% FFA. TLC plating showed that the distillate fraction was exclusively mono-esters with the remaining portion being FFAs.

A second distillation was tested to further reduce the FFA from the distillate fraction of mono-esters which still had 0.9% FFA in order to further purify and remove the FFA. This distillation was performed at 155° C. at 210° C., 230 rpm rotor speed, and about 0.024 mb vacuum. The distillation yielded 2 fractions. The retentate fraction was 77.4 g of mono-esters with 0.1% FFA and the distillate fraction was 16 g of mostly mono-esters with 7.8% FFAs.

### EXAMPLE 8

Preparation of mono- and di-esters of palmitic acid.

The palmitic acid was 95% pure and sourced from Sigma-Aldrich, St. Louis, Mo. The reaction mixture included 256 g (1 mole) of palmitic acid, 40 g of LIPOZYME 435 brand lipase, and 146 g (1 mole) of isosorbide were used. The reaction was carried out at 80° C. substantially as described in Example 3. This reaction achieved 0.5% FFA in 3 hours.

The reaction mixture was separated by filtration. A TLC plate was prepared as described in Example 1 to test for the presence of di- and mono-esters. Mono-palmitic esters and di-palmitic esters were visible on the TLC plate. The reaction mixture (275 g) was processed by short-path distillation under the same conditions as described in Example 7. The retentate fraction of purified di-esters yielded 89.0 g of material that had 0.9% FFAs. The retentate fraction showed no presence of mono-esters. A second distillation was done on the distillate fraction at 155° C. which yielded 74 g of purified mono-esters which had 0.1% FFA remaining There were 21.7 g of a FFA fraction which had a portion of mono-esters present and visible on the TLC plate. Slowing down the flow rate may help improve the separation.

### EXAMPLE 9

Esterification with caprylic acid.

The caprylic acid was 98% pure. The reaction mixture included 72 g (0.5 mol) of caprylic acid, 14.5 g of LIPOZYME 435 brand lipase, and 73 g (0.5 mol) of isosorbide. The reaction was carried out at 70° C. substantially as described in Example 3. This reaction achieved 28% FFA in 4.5 hours.

### EXAMPLE 10

Esterification with capric acid.

The capric acid was 98% pure and sourced from Sigma-Aldrich, St. Louis, MO. The reaction mixture included 86.3 g (0.5 mol) of capric acid, 15.9 g of LIPOZYME 435 brand lipase, and 73 g (0.5 mol) of isosorbide. The reaction was carried out at 70° C. substantially as described in Example 3. This reaction achieved 0.9% FFA in 3 hours.

A larger reaction was performed with 172.6 g capric acid, 32 g of LIPOZYME 435 brand lipase, and 146 g of isosorbide. The reaction was carried out at 70° C. substantially as described in Example 3. This reaction achieved 0.8% FFA in 3 hours. The reaction mixture of the larger batch was separated by filtration and 300 g of the filtered material was processed by short-path distillation.

The separation of the di-capric esters was performed at 155° C. and the second distillation used to purify the mono-capric esters was performed at 95° C. The fraction of purified di-esters resulted in 73 g of material that had 0.9% FFAs and TLC analysis showed no presence of mono-esters. The second distillation yielded 142.5 g of purified mono-esters which has 0.3% FFA.

### EXAMPLE 11

Esterification with lauric acid.

The lauric acid was 98% pure and sourced from TCI America, Portland, OR. The reaction mixture included 200 g (1 mole) of lauric acid, 34.6 g of LIPOZYME 435 brand lipase, and 146 g (1 mole) of isosorbide. The reaction was carried out at 80° C. substantially as described in Example 3. This reaction achieved 0.1% FFA in 3 hours. The reaction mixture was separated by filtration and 250 g of the filtered material was processed by short-path distillation.

The distillation temperatures were 180° C. and 125° C. The fraction of purified di-lauric esters yielded 78.3 g of material that had 0.9% FFA and the TLC plate showed no presence of mono-esters. The second distillation yielded 45.6 g of purified mono-lauric esters which had 0.2% FFA.

### EXAMPLE 12

Esterification with behenic acid.

The behenic acid was 95% pure and sourced from TCI America, Portland, OR. Behenic acid has a melting point of about 80° C. A first reaction mixture included 85.1 g (0.25 mol) of behenic acid, 12.1 g of LIPOZYME 435 brand lipase, and 36.5 g (0.25 mol) of isosorbide. The reaction was carried out at 82.5° C. substantially as described in Example 3. After 4 hours, there was 2.7% FFA remaining

A second reaction mixture included 170 g (0.5 mol) of behenic acid, 243 g of LIPOZYME 435 brand lipase, and 73 g (0.5 mol) of isosorbide. The reaction was carried out at 82.5° C. substantially as described in Example 3. After 5 hours, there was 2.8% FFA remaining A short path still temperature of 210° C. was used to separate the di-esters from the mono-esters. The distillation yielded 94.8 g of purified di-behenic esters with no FFA, and the mono-esters fraction has 12% FFA.

### EXAMPLE 13

Esterification of oleic acid and isoiodide.

50.8 g (180 mmol) of practical grade oleic acid, 7.54 g of LIPOZYME 435 brand lipase, and 24.6 g (178 mmol) of purified isoiodide (more than 99% pure) was reacted at 80° C. substantially as described in Example 3. The progress of the reaction is shown in Table 1.

TABLE 1
Reaction kinetics of rate of formation of isoiodide oleate.

| Reaction time (hr) | % free fatty acids |
|---|---|
| 1 | 62 |
| 2 | 40 |
| 3 | 23 |
| 4 | 12 |
| 5 | 4.9 |
| 6 | 1.3 |

The reaction mixture was tested by TLC plate and both di- and mono-esters were visible. The reaction with isoiodide, which has two “exo” hydroxyl groups, was lower than the reaction with isosorbide, which has “exo” and “endo” hydroxyl groups.

### EXAMPLE 14

Esterification of oleic acid and isomannide.

33.9 g (120 mmol) of oleic acid, 5 g of LIPOZYME 435 brand lipase, and 16.4 g (112 mmol) of purified isomannide (more than 95% pure) was reacted at 80° C. substantially as described in Example 3. The progress of the reaction is shown in Table 2.

TABLE 2
Reaction kinetics of rate of formation of isomannide oleate.

| Reaction time (hr) | % free fatty acids |
|---|---|
| 0.5 | 13 |
| 1 | 2.9 |
| 1.5 | 1.4 |
| 2 | 0.9 |

The reaction mixture was tested by TLC plate and both di- and mono-esters were visible. Reaction with isomannide, which has two “endo” hydroxyls, was faster than the reaction with isoiodide.

### EXAMPLE 15

Use of mono- and di-ester derivatives of isosorbide for gelation of vegetable oils.

A weighed amount of isosorbide esters (mono- or di-ester derivatives with C 16, C18, C18:1, and C22 fatty acids) and an organic liquid (oils) were placed in a scintillation vial. The vial was heated with continuous agitation until the solid material had dissolved in the organic phase. The solution was allowed to cool to room temperature. The state of the isosorbide ester-organic liquid mixture resulting from its cooling was analyzed for its mechanical strength (strength and yield stress) by performing dynamic oscillatory rheology. The liquid was said to be gelled when: i) the contents in the vial did not exhibit any flow under the gravity upon inversion of the vial, or ii) the storage modulus (G′) was greater than the viscous modulus (G″) by at least a factor of three.

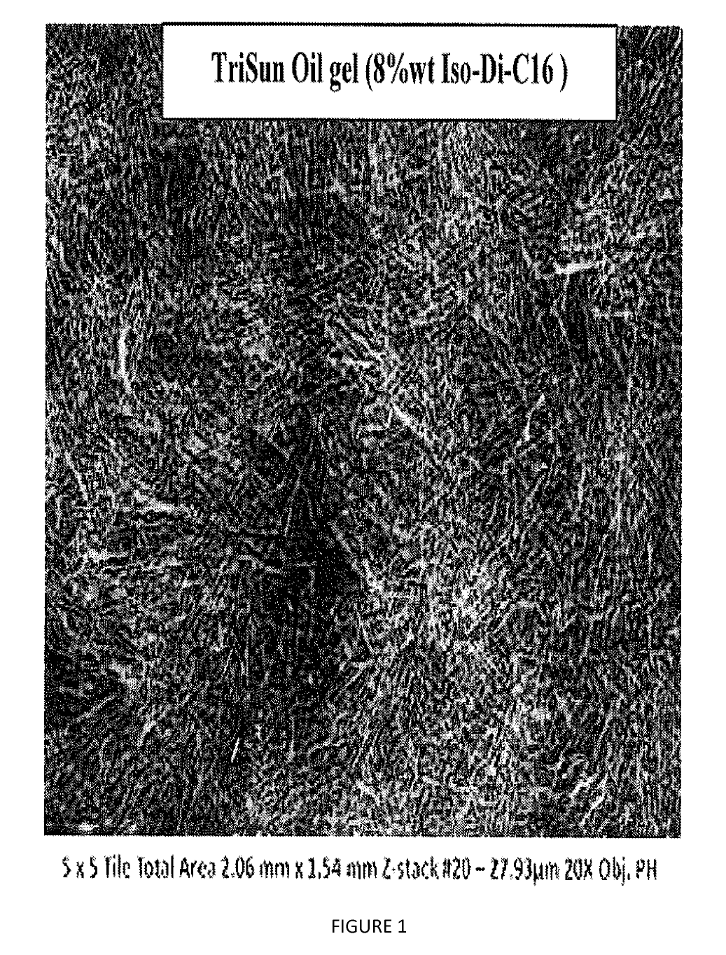

When a heterogeneous mixture of an ester of 1,4:3,6-dianhydrohexitols and an organic liquid were heated, the ester derivatives either dissolve in the organic liquid or become finely dispersed. Upon cooling of the heterogeneous mixture, the solubility of the gelator molecules (i.e., the 1,4:3,6-dianhydrohexitols) progressively decreases with the decreased temperature and at a certain point, a supersaturated state of the heterogeneous mixture is achieved. Further decreases in temperature lead to precipitation and the formation of nanoscale nuclei. Around the nuclei, the gelator molecules self-assemble to form liquid crystalline lamellar structures, 1-D micrometer long fibrous aggregates. Non-covalent forces such as H-bonding, van der Waal's forces, London dispersion forces, and others were found to drive the self-assembly process. A morphological analysis of the mixture using optical microscopy revealed the presence of a network having micron-size fibrous aggregates as shown in FIG. 1. Such aggregates further entangle via non-covalent interactions to form a volume filling 3-D network, which in turn entraps liquid molecules via surface tension and capillary action, thus structuring or gelling the organic phase.

The ability of various isosorbide ester derivatives to gelate vegetable oil was determined and compared to a commercially available sugar ester, i.e., sorbitan monoalkylates (represented by Span #). The various isosorbide ester derivatives were used at a concentration of 8% by weight on an oil basis. The various gelators that were used and their gelation ability is shown in Table 3. The nomenclature of the gelator starts with the type of 1,4:3,6-dianhydrohexitols, followed by the number of fatty acid chains and the type of fatty acid. For instance, iso-mono-C22 refers to an isosorbide monoester of behenic acid (isosorbide monobehenate) and iso-di-C22 refers to isosorbide diester of behenic acid (isosorbide dibehenate). State refers to the state of the isosorbide ester/organic liquid mixture after cooling. The average value of elastic modulus in the linear viscoelastic region and is a measure of the strength of a gel. The average value of complex modulus in the linear viscoelastic regions and is a measure of the strength of a gel. The yield point (YP) is the applied oscillatory stress value at G′/G″ crossover and signifies the point at which the 3-D network breaks and the mixture starts flowing like a viscous fluid.

TABLE 3
Vegetable oil gelation capabilities of different
isosorbide ester derivatives.

| Vegetable Oil | Gelator | State | G′ (Pa) | G* (Pa) | YP (Pa) |
|---|---|---|---|---|---|
| canola | Iso-mono-C22 | fluid | 16.54 | 21.05 | 0.38 |
| canola | Iso-di-C22 | strong gel | 53094 | 54432 | 116.76 |
| canola | Iso-mono-C18 | weak gel | 540 | 588 | 1.5 |
| canola | Iso-di-C18 | strong gel | 71878 | 74465 | 49.2 |
| canola | Span-60 | fluid | 33.8 | 34.8 | 1 |
| canola | Iso-mono-C16 | weak gel | 49.2 | 54 | 0.31 |
| canola | Iso-di-C16 | strong gel | 47530 | 49362 | 24.05 |
| canola | Span-40 | fluid | 18.28 | 18.72 | 1.15 |
| high oleic | Iso-mono-C22 | fluid | 40.16 | 44.85 | 2.42 |
| canola | | | | | |
| high oleic | Iso-di-C22 | strong gel | 31907 | 32448 | 57.08 |
| canola | | | | | |
| high oleic | Iso-mono-C18 | weak gel | 1708 | 1810 | 2.8 |
| canola | | | | | |
| high oleic | Iso-di-C18 | strong gel | 15195 | 15401 | 15.81 |
| canola | | | | | |
| high oleic | Span-60 | fluid | 21.4 | 22 | 1 |
| canola | | | | | |
| high oleic | Iso-mono-C16 | weak gel | 52.11 | 59.96 | 0.45 |
| canola | | | | | |
| high oleic | Iso-di-C16 | strong gel | 19431.5 | 20131.84 | 12.2 |
| canola | | | | | |
| high oleic | Span-40 | fluid | 42.95 | 46.34 | 1.38 |
| canola | | | | | |
| soy | Iso-mono-C22 | fluid | 22.64 | 26.74 | 0.35 |
| soy | Iso-di-C22 | strong gel | 56155 | 58985 | 65.48 |
| soy | Iso-mono-C18 | weak gel | 385.8 | 419.5 | 1.22 |
| soy | Iso-di-C18 | strong gel | 78326.5 | 106523 | 41.07 |
| soy | Span-60 | fluid | 21.04 | 21.52 | 0.97 |
| soy | Iso-mono-C16 | weak gel | 75.54 | 85.42 | 0.44 |
| soy | Iso-di-C16 | strong gel | 34754.2 | 36775.9 | 15.89 |
| soy | Span-40 | fluid | 24.8 | 26 | 1.17 |
| high oleic soy | Iso-mono-C22 | fluid | 40.15 | 46.04 | 0.65 |
| high oleic soy | Iso-di-C22 | strong gel | 53905 | 54462 | 89.66 |
| high oleic soy | Iso-mono-C18 | weak gel | 209.4 | 228.9 | 0.95 |
| high oleic soy | Iso-di-C18 | strong gel | 29156.5 | 29451.1 | 18.87 |
| high oleic soy | Span-60 | fluid | 27.97 | 28.86 | 1.7 |
| high oleic soy | Iso-mono-C16 | weak gel | 23.51 | 31.06 | 0.1 |
| high oleic soy | Iso-di-C16 | strong gel | 39482 | 40320.9 | 20.85 |
| high oleic soy | Span-40 | fluid | 23.2 | 24.5 | 1.38 |
| mid oleic | Iso-mono-C22 | fluid | 32.65 | 38.14 | 1.08 |
| sunflower | | | | | |
| mid oleic | Iso-di-C22 | strong gel | 46139 | 46839 | 59.33 |
| sunflower | | | | | |
| mid oleic | Iso-mono-C18 | weak gel | 631.2 | 682.2 | 1.64 |
| sunflower | | | | | |
| mid oleic | Iso-di-C18 | strong gel | 51961.9 | 53192 | 33.13 |
| sunflower | | | | | |
| mid oleic | Span-60 | fluid | 29.9 | 30.5 | 1.25 |
| sunflower | | | | | |
| mid oleic | Iso-mono-C16 | weak gel | 61.7 | 70.27 | 0.17 |
| sunflower | | | | | |
| mid oleic | Iso-di-C16 | strong gel | 3062 | 3215.7 | 2.08 |
| sunflower | | | | | |
| mid oleic | Span-40 | fluid | 20.4 | 21 | 1.42 |
| sunflower | | | | | |
| high oleic | Iso-mono-C22 | fluid | 28.12 | 32.32 | 1.22 |
| sunflower | | | | | |
| high oleic | Iso-di-C22 | strong gel | 53493 | 54267 | 80.70 |
| sunflower | | | | | |
| high oleic | Iso-mono-C18 | weak gel | 83.02 | 93.70 | 0.46 |
| sunflower | | | | | |
| high oleic | Iso-di-C18 | strong gel | 42138.5 | 43098 | 20.09 |
| sunflower | | | | | |
| high oleic | Span-60 | fluid | 27.14 | 28 | 0.4 |
| sunflower | | | | | |
| high oleic | Iso-mono-C16 | weak gel | 48.3 | 53.72 | 0.28 |
| sunflower | | | | | |
| high oleic | Iso-di-C16 | strong gel | 47444.5 | 48229.5 | 24.17 |
| sunflower | | | | | |
| high oleic | Span-40 | fluid | 23.1 | 23.8 | 1.36 |
| sunflower | | | | | |

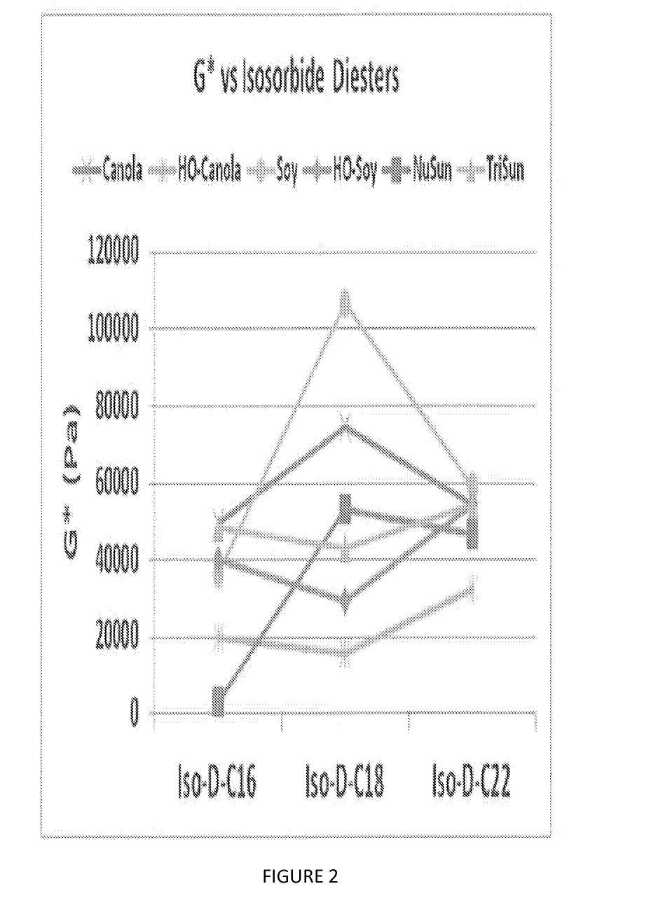

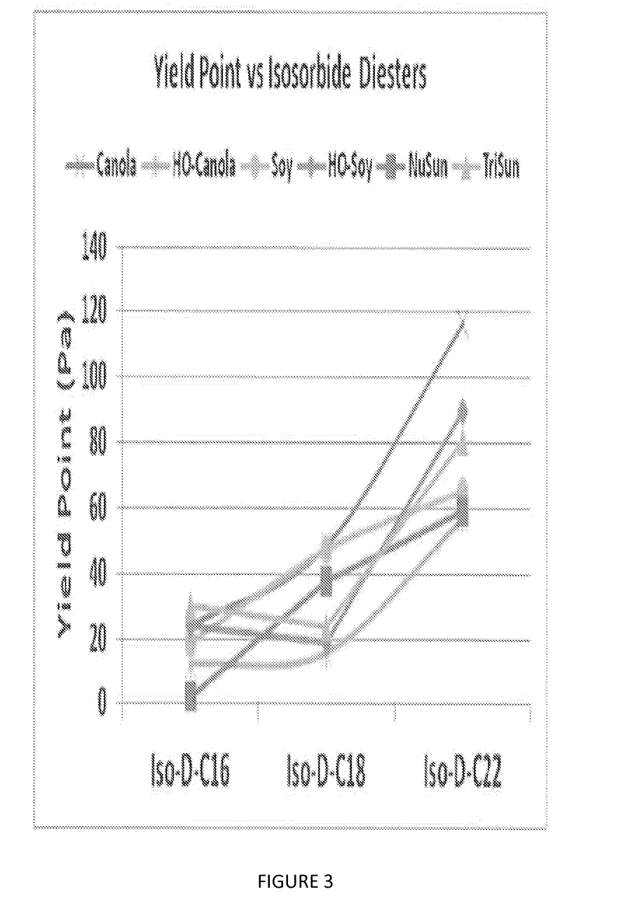

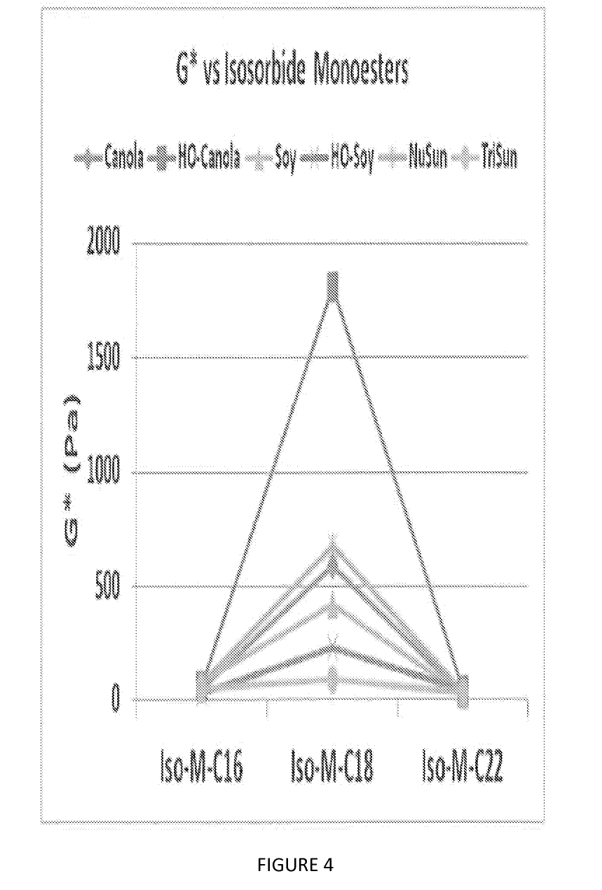

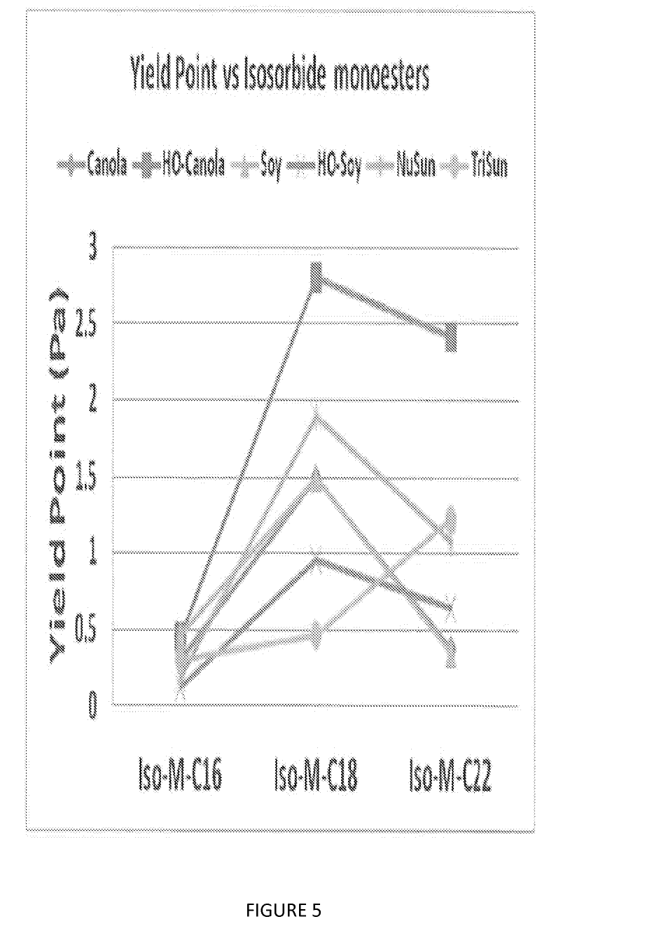

FIG. 2 shows the effect of the type of diesters on G*, the average value of complex modulus. FIG. 3 shows the effect of the type of diesters on the Yield Point. FIG. 4 shows the effect of the type of monoesters on G*, the average value of complex modulus. FIG. 5 shows the effect of the type of monoesters on the yield point.

Table 3 shows that irrespective of the type of oil and for a given fatty acid chain length, the isosorbide diesters demonstrated a better gelation efficiency as compared to the analogous isosorbide monoesters. The isosorbide diester-based gels exhibited higher gel strength (G*) and yield point (YP). The presence of 2 fatty acids on the isosorbide presumably increases inter-amphiphilic association (i.e., gelator-gelator) and with oil molecules (i.e., gelator-oil). The enhancement in the degree of intermolecular interactions leads to a development of a stronger network, which in turn improves the mechanical properties of the gels.

The effect of the acyl chain length attached to the isosorbide on gelation was evident from Table 3. Generally, the gel strength (i.e., G* and YP) was found to increase when the acyl chain length of the fatty acid was increased from C16 to C18. In Table 3, the isosorbide monoester C18 (isosorbide monostearate) was found to have the optimum gelation efficiency and the optimum chain length for the isosorbide monoester-based gelators was between 18-20. The chain length of 18-20 of the isosorbide monoesters possessed a critical balance between hydrophilic (i.e., the sugar head group) and hydrophobic (i.e., the acyl chain length) interactions to exhibit efficient gelation efficiency.

With regards to the diester derivatives and depending on the type of oils (i.e., conventional oils v. high oleic oils), two trends were observed in Table 3. With the conventional oils, the gelation efficiency of the diester derivatives followed the trend similar to that of the monoester derivatives where the C18 derivative (i.e., isosorbide distearate) was found to exhibit optimum gelation efficiency and the strength of the gels (i.e., G* and YP) was maximum. On the other hand, for high oleic oils, the isosorbide esters exhibited the opposite trend where the gel strength (i.e., G* and YP) was found to decrease when the acyl chain length was increased from C16 to C18. However, the gel strength did increase when the acyl chain length was increased to C22. This trend appears to indicate that the polarity of the oil plays an important role in controlling the self-assembling tending of the isosorbide derivatives.

The effect of the degree of unsaturation on fatty acid chains (i.e., C18 v. C18:1) on gelation efficiency was also evident from Table 3. Typically, the introduction of unsaturations (i.e., pi bonds) in fatty acid chains of amphiphiles is known to affect the self-assembling properties and gelation capabilities of an amphiphile. Similarly, there appeared to be a considerable difference in gelation capabilities upon changing the fatty acid chain from C18 (stearic acid) to C18:1 (oleic acid) on the isosorbide-based gelators.

Unlike isosorbide monostearate, isosorbide monooleate was not able to gel any of the oils, but was able to partially self-assemble in the oils and increase the viscosity of the oils.

Unlike isosorbide distearate, the isosorbide dioleate was liquid. The isosorbide dioleate could not gel any of the tested oils, but was infinitely miscible with the oils and infinitely miscible with lecithin samples. Thus, isosorbide dioleate would probably be a good plasticizer.

The introduction of unsaturation generally decreased the gelation efficiency of mono- and di-ester isosorbide derivatives. The unsaturations decrease the linearity in the acyl chain, which in turn decreases the lateral interaction of acyl chain and, thus, the self-assembling tendency of amphiphiles.

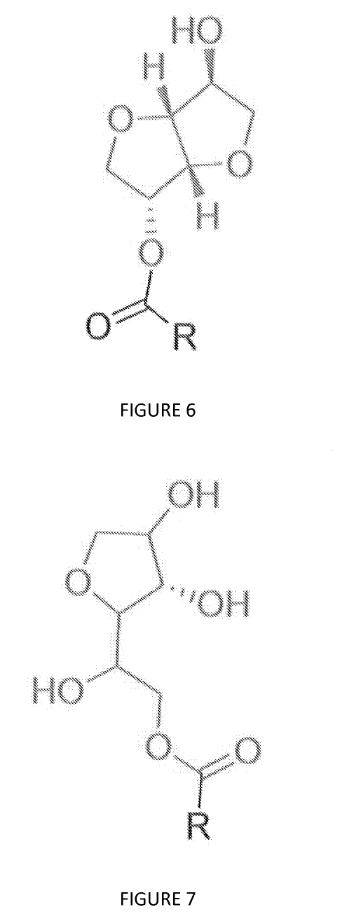

The effect of different head groups on gelation efficiency was determined. A representative molecular structure of a isosorbide monoester is shown in FIG. 6 and a representative molecular structure of a sorbitan monoester is shown in FIG. 7.

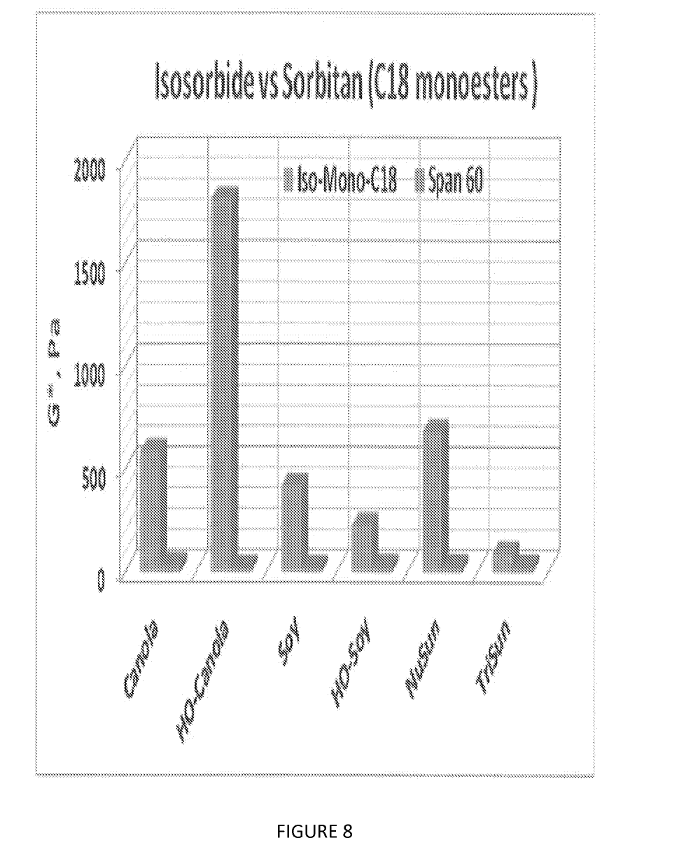

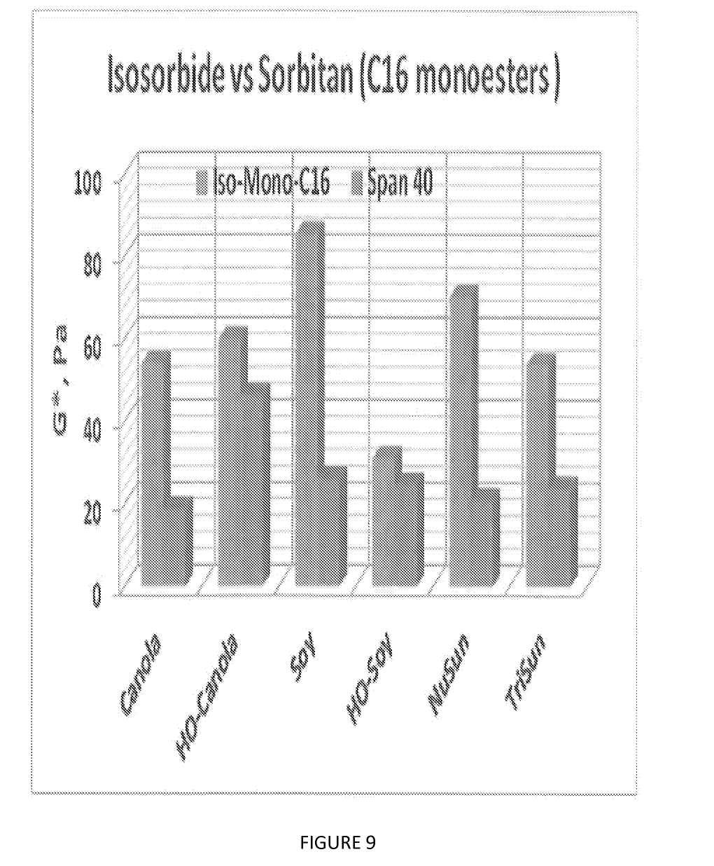

To analyze the effect of the head group structure on gelation efficiency, isosorbide monoester derivatives were compared to analogous sorbitan monoester derivatives. Isosorbide monostearate (Iso-mono-C18) was compared to sorbitan monostearate (span 60) and isosorbide monopalmitate (Iso-mono-C16) was compared to sorbitan monopalmitate (span 40). As evident from Table 3 and FIGS. 8 and 9, isosorbide monoester derivatives exhibit better self-assembling tendency compared to sorbitan monoesters as G* and YP were consistently higher for the isosorbide monoesters.

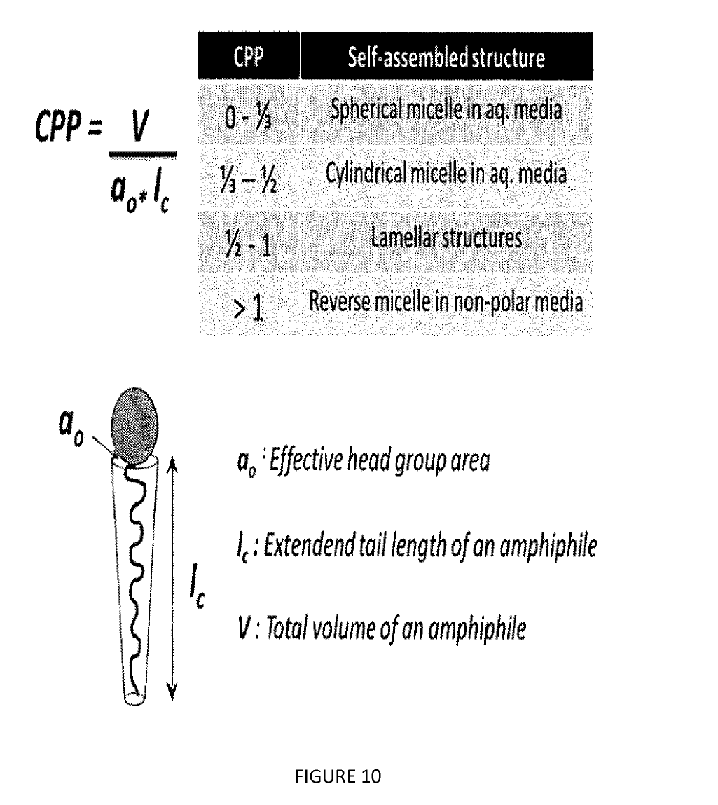

The difference in self-assembling tendency can be attributed to the structure of the head groups and the critical packing parameter (CPP), described in FIG. 10. The CPP determines the preferred association structures assumed for each molecular shape. CPP is inversely proportional to the area of head group. The difference in the molecular structure of isosorbide and sorbitan has a profound effect on the nature of self-assembled structures of their ester derivatives and, thus, their gelation efficiency. Also, the area of isosorbide is relatively smaller than sorbitan and, therefore, the ester derivatives of isosorbide are believed to facilitate formation of stronger lamellar structure as compared to sorbitan ester derivatives. For the same reason, the fatty acid chain length adds to the contribution from the head group in increasing the gel strength. Thus, the isosorbide monoester would form a stronger gel than its analogous sorbitan monoester. Similarly, the isosorbide diester would form a stronger gel than the sorbitan trimester derivative with the same chain length of fatty acids.

While this invention has been particularly shown and described with references to exemplary embodiments thereof, it will be understood by those skilled in the art that various changes in form and details may be made therein without departing from the scope of the invention encompassed by the appended claims.

What is claimed is:

1. A structured non-polar liquid oleogel, comprising:
a non-polar liquid; and
a derivative of an anhydrohexitol.

2. The structured non-polar liquid oleogel of claim 1, wherein the derivative of the anhydrohexitol is selected from the group consisting of an ester of the anhydrohexitol, an amide of the anhydrohexitol, an ether of the anhydrohexitol, a dimer of the anhydrohexitol, and combinations of any thereof.

3. (canceled)

4. The structured non-polar liquid oleogel of claim 1, wherein the derivative of the anhydrohexitol is a self-assembled, liquid crystalline structure in the non-polar liquid.

5. The structured non-polar liquid oleogel of claim 1, further comprising an emulsifier.

6. (canceled)

7. The structured non-polar liquid oleogel of claim 1, wherein the derivative of the anhydrohexitol is present at an amount of between 0.1-15% by weight.

8. (canceled)

9. The structured non-polar liquid oleogel of claim 1, wherein the non-polar liquid is present at an amount of between 80-99.9% by weight.

10-13. (canceled)

14. The structured non-polar liquid oleogel of claim 1, wherein the anhydrohexitol is 1,4:3,6-dianhydrohexitol.

15-17. (canceled)

18. The structured non-polar liquid oleogel of claim 1, wherein the structured non-polar liquid melts at a temperature of at least 40° C. and re-forms at a temperature of less than 40° C.

19-20. (canceled)

21. The structured non-polar liquid oleogel of claim 1, wherein the non-polar liquid is selected from the group consisting of soybean oil, canola oil, sunflower oil, olive oil, sesame oil, grapeseed oil, rapeseed oil, linseed oil, neem oil, liquid paraffin, corn oil, soy polyols, biodiesel, diesel oil, a lubricant grease, a modified vegetable oil, a petroleum based solvent, mineral oil, esters of any thereof, and combination of any thereof.

22-23. (canceled)

24. The structured non-polar liquid oleogel of claim 1, further comprising a structuring agent.

25. The structured non-polar liquid oleogel of claim 24, wherein the structuring agent is selected from the group consisting of ethyl cellulose, hydroxyl-stearic acid, sterols, proteins, emulsifiers, waxes, and combinations of any thereof.

26. A method of producing a structured non-polar liquid oleogel comprising a non-polar liquid and a derivative of an anhydrohexitol, the method comprising:
mixing the derivative of the anhydrohexitol with the non-polar liquid, thus producing a mixture; and
heating the mixture.

27-28. (canceled)

29. The method of claim 26, further comprising adding an emulsifier to the mixture.

30. A process for producing an ester of an anhydrohexitol in the a structured non-polar liquid oleogel comprising a non-polar liquid and a derivative of the anhydrohexitol wherein the derivative of the anhydrohexitol is the ester of the andydrohexitol, the process comprising:
combining the anhydrohexitol with a carboxylic acid, thus producing a mixture; and
placing the mixture in contact with an enzyme, such that an ester bond is formed between the anhydrohexitol and the carboxylic acid.

31. The process of claim 30, wherein the enzyme is lipase.

32. The process of claim 30, further comprising combining an organic solvent with the mixture.

33. The process of claim 30, further comprising removing water from the mixture, such that ester bond formation occurs in the absence of water.

34. The process of any one of claim 30, wherein the anhydrohexitol is selected from the group consisting of isosorbide, isomannide, isoiodide, and combinations of any thereof.

35. The process of any one of claim 30, wherein the anhydrohexitol is 1,4:3,6-dianhydrohexitol.

36. The process of any one of claim 30, wherein the carboxylic acid is a fatty acid.

37-40. (canceled)
